(19) United States
(12) Patent Application Publication
(10) Pub. No.: US 2015/0175505 A1
(43) Pub. Date: Jun. 25, 2015

(54) METHODS AND SYSTEMS FOR ISOMERIZING PARAFFINS

(71) Applicant: UOP LLC, Des Plaines, IL (US)

(72) Inventors: Bryan K. Glover, Algonquin, IL (US); Rajaraman Panchapakesan, Gurgaon (IN); Pankaj Kumar Singh, New Delhi (IN)

(73) Assignee: UOP LLC, Des Plaines, IL (US)

(21) Appl. No.: 14/134,116

(22) Filed: Dec. 19, 2013

(51) Int. Cl. C07C 5/27; C07C 5/31

(52) U.S. Cl. CPC C07C 5/2729; C07C 5/31

(57) ABSTRACT

Disclosed is a method and system for isomerizing paraffins to improve the isomerate yield, to minimize catalyst content, and to reduce the pressure drop observed in the isomerization reactor. In one embodiment, a method for isomerizing paraffins includes providing a hydrocarbon stream including linear paraffin compounds and passing the hydrocarbon stream to a first lead/lag isomerization reactor pair to isomerize a portion the linear paraffin compounds to branched paraffin compounds. A second portion of the linear paraffin compounds remain as linear paraffins compounds. The method further includes deisohexanizing the branched and linear paraffin compounds to form an overhead product stream, a bottom product stream, and a side-cut stream comprising the linear paraffin compounds and passing the side-cut stream to a second lead/lag isomerization reactor pair to isomerize the linear paraffin compounds to branched paraffin compounds.

### TECHNICAL FIELD

The present disclosure generally relates to methods and systems for isomerizing paraffins. More particularly, the present disclosure relates to methods and systems for isomerizing linear and cyclo-paraffins employing a dual isomerization reactor zone configuration.

### BACKGROUND

Processes for the isomerization of normal and cyclo-paraffins into more highly branched paraffins are widely practiced. Particularly important commercial isomerization processes are used to increase the branching, and thus the octane value of refinery streams containing paraffins of 4 to 8, and especially 5 and 6, carbon atoms. The isomerate is typically blended with a refinery reformer effluent to provide a blended gasoline mixture having a desired research octane number (RON).

The isomerization process proceeds toward a thermodynamic equilibrium. Hence, the isomerate will still contain normal paraffins and cyclo-paraffins that have low octane ratings and thus detract from the octane rating of the isomerate. Provided that adequate high octane blending streams such as alkylate and reformer effluent is available and that gasolines of lower octane ratings, such as 85 and 87 RON, are in demand, the presence of these normal and cyclo-paraffins in the isomerate has been tolerated.

Where circumstances demand higher RON isomerates, however, the isomerization processes have been modified by separating the normal and cyclo-paraffins from the isomerate and recycling them to the isomerization reactor. Thus, not only are un-branched paraffins that detract from the octane rating removed from the isomerate, but also their return to the isomerization reactor increases the portion of the feed converted to the more highly desired branched paraffins.

The most frequently practiced isomerization processes that recycle normal paraffins use a deisohexanizer. A deisohexanizer is one or more distillation columns where an overhead containing branched C₆ paraffins such as dimethylbutanes (2,2-dimethylbuthane and 2,3-dimethylbutane) and lighter components is obtained as the isomerate product for, e.g., blending for gasolines, and a side-stream containing normal hexane and similarly boiling components such as methylpentanes (2-methylpentane and 3-methylpentane), methylcyclopentane, and cyclohexane are recycled back to the isomerization reactor.

However, it has been observed that when normal and cyclo-paraffins are recycled back to the isomerization reactor, there is an undesirably low product yield due to the requirement to process the recycle stream in addition to the incoming feed stream. Further, it has been observed that the catalyst quantity requirement in the isomerization is also undesirably high, again due to the requirement to process the recycle stream in addition to the incoming feed stream.

Accordingly, it is desirable to provide economically viable, and simple to operate processes and system to enhance the octane rating of isomerized paraffins. Additionally, it is desirable to provide such methods and systems that minimize the catalyst content required for the isomerization reactor and increase the isomerate product yield. Furthermore, other desirable features and characteristics of the present disclosure will become apparent from the subsequent detailed description and the appended claims, taken in conjunction with the accompanying drawings and this background.

### BRIEF SUMMARY

The present disclosure generally provides methods and systems for isomerizing paraffins with increased isomerate yield and reduced catalyst content. In one exemplary embodiment, disclosed is a method for isomerizing paraffins that includes providing a hydrocarbon stream including linear paraffin compounds and passing the hydrocarbon stream to a first lead/lag isomerization reactor pair to isomerize a portion the linear paraffin compounds to branched paraffin compounds. A second portion of the linear paraffin compounds remain as linear paraffins compounds. The method further includes deisohexanizing the branched and linear paraffin compounds to form an overhead product stream, a bottom product stream, and a side-cut stream comprising the linear paraffin and cyclo-paraffin compounds and passing the side-cut stream to a second lead/lag isomerization reactor pair to isomerize the linear paraffin compounds to branched paraffin compounds and convert the cyclo-paraffin compounds to branched paraffin compounds.

In another exemplary embodiment, disclosed is a system for isomerizing paraffins that includes a first lead/lag reactor pair that receives a hydrocarbon stream including linear paraffin compounds and generates an effluent including linear paraffin compounds, branched paraffin compounds, and cyclo-paraffin compounds, a deisohexanizer that receives the effluent from the first lead/lag reactor pair and generates an overhead product stream, a bottoms product stream, and a side-cut stream including the linear paraffin compounds, and a second lead/lag reactor pair that receives the side-cut stream and generates branched paraffin compounds.

### BRIEF DESCRIPTION OF THE DRAWING

The present embodiments will hereinafter be described in conjunction with the following drawing FIGURE, wherein like numerals denote like elements, and wherein:

FIG. 1 is a process flow diagram illustrating a method implemented on a paraffin isomerization system in accordance with various embodiments of the present disclosure.

### DETAILED DESCRIPTION

The following detailed description is merely exemplary in nature and is not intended to limit the application and uses of the embodiments described. Furthermore, there is no intention to be bound by any theory presented in the preceding background or the following detailed description.

The present disclosure provides methods and systems for isomerizing paraffins. The embodiments described herein employ a novel combination of two separate isomerization reactor zones to convert linear paraffins, such as C₅ and C₆ linear paraffins and cyclo-paraffins, into branched paraffins. A first reactor zone isomerizes a portion of the linear land cyclo-paraffins contained in a feed stream that is fed thereto. The effluent from the first reactor zone optionally passes through a separator to remove and recycle hydrogen gas, a stabilizer to remove light hydrocarbons, such as C₄ and lighter hydrocarbons and hydrogen gas, and a deisohexanizer. The deisohexanizer separates the effluent into lower boiling hydrocarbons, a side-cut stream containing non-isomerized linear paraffins and cyclo-paraffins, and higher boiling hydrocarbons, as will be described in greater detail below. The side-cut stream is passed to a second reactor zone that isomerizes the linear paraffins and the cyclo-paraffins in the side-cut stream, i.e., those that were not isomerized in the first reactor zone. The effluent form the second reactor zone joins with the effluent from the first reactor zone prior to its entry (optionally) into the separator or the stabilizer. In this manner, a paraffin isomerization method and system is provided that maximizes the isomerate yield and that minimizes the catalyst quantity required for the isomerization reactors.

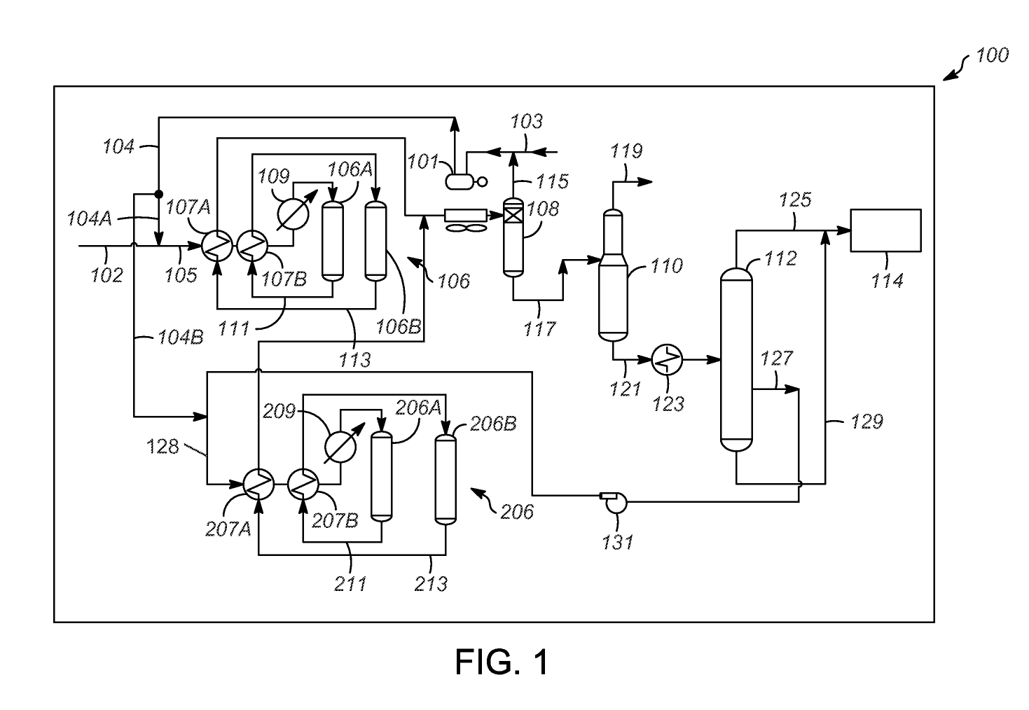

FIG. 1 is a process flow diagram illustrating a method implemented on a paraffin isomerization system 100 in accordance with various embodiments of the present disclosure. As shown in FIG. 1, a linear and cyclo-paraffin-containing feedstock is supplied to system 100 via line 102. Furthermore, hydrogen gas (H₂) is provided via line 104 and 104A. Any suitable paraffin-containing feedstock may be used in the processes of the present disclosure. For example, naphtha feedstocks are the most often used as the feedstocks to isomerization processes. Naphtha feedstocks may include paraffins, naphthenes, and aromatics, and may include small amounts of olefins, boiling within the gasoline range. Feedstocks that may be utilized include straight-run naphthas, natural gasoline, synthetic naphthas, thermal gasoline, catalytically cracked gasoline, partially reformed naphthas, or raffinates from extraction of aromatics. The feedstock may be encompassed by the range of a full-range naphtha, or a naphtha having a boiling range from about 0° C. to about 230° C. In one embodiment, the feedstock is a light naphtha having an initial boiling point of about 10° C. to about 65° C. and a final boiling point from about 75° C. to about 110° C.

Naphtha feedstocks may contain small amounts of sulfur compounds amounting to less than about 10 mass parts per million (mppm) on an elemental basis. For example, the naphtha feedstock may have been prepared from a feedstock by a conventional pretreating step such as hydrotreating, hydrorefining, or hydrodesulfurization to convert such contaminants as sulfurous, nitrogenous and oxygenated compounds to H₂S, NH₃ and H₂O, respectively, which can be separated from hydrocarbons by fractionation. This conversion may employ a catalyst known to the art including an inorganic oxide support and metals selected from Groups VIB(IUPAC 6) and VIII(IUPAC 9-10) of the Periodic Table. Water can act to attenuate catalyst acidity by acting as a catalyst base, and sulfur temporarily deactivates the catalyst by platinum poisoning. Feedstock hydrotreating as described hereinabove may reduce water-generating oxygenates and deactivating sulfur compounds to suitable levels, and other means such as adsorption systems for the removal of sulfur and water from hydrocarbon streams generally are not required. It is within the ambit of the present disclosure that this optional pretreating step be included in the present process combination.

The principal components of the feedstock according to some embodiments are cyclic and acyclic paraffins having from 4 to 8 carbon atoms per molecule (C₄ to C₈), such as C₅ and C₆, and smaller amounts of aromatic and olefinic hydrocarbons also may be present. In some embodiments, the concentration of C₇ and heavier components is less than about 20 mass-percent of the feedstock, and the concentration of C₄ and lighter components is less than about 20 mass-percent, for example less than about 10 mass-percent, of the feedstock. The mass ratio of C₅ to C₆ components may be from about 1:10 to about 10:1. In alternative embodiments, the feedstock may include solely C₅ or solely C₆ compounds.

Although there are no specific limits to the total content in the feedstock of cyclic hydrocarbons, the feedstock in some embodiments contains between about 2 and about 40 mass-percent of cyclics including naphthenes and aromatics. The aromatics contained in the naphtha feedstock, although generally amounting to less than the alkanes and cycloalkanes, may include from about 2 to about 20 mass-percent. Benzene may be the principal aromatics constituent of the feedstock, optionally along with smaller amounts of toluene and higher-boiling aromatics within the boiling ranges described above.

In general, linear paraffins may constitute at least about 15, for example from about 40, such as at least about 50, mass-percent to essentially all of the feedstocks used in the processes of the present disclosure. For naphtha feedstocks, linear paraffins may be present in amounts of at least to about 50, for example from about 50 to about 90, mass-percent. The mass ratio of non-linear paraffins to linear paraffins in the feedstocks may be less than about 1:1, for example from about 0.1:1 to about 0.95:1. Non-linear paraffins include branched acyclic paraffins and substituted or unsubstituted cycloparaffins. Other components such as aromatics and olefinic compounds may also be present in the feedstocks as described above.

The feed streams 102, 104A are thereafter combined into a feed stream 105, according to one exemplary embodiment. Feed stream 105 passes through two heat exchangers 107A, 107B (described in greater detail below) and a heater 109 to increase the temperature of the feed stream 105 to a suitable isomeration reactor temperature, as will be described in greater detail below. Upon passing through heat exchangers 107A, 107B, and heater 109, the feed stream 105 enters a first isomerization reactor zone 106, and particularly lead isomerization reactor 106A of a lead/lag isomerization reactor pair. While two isomerization reactors are shown in the first reactor zone, it should be appreciated that one, three, or more reactors may be provided in alternative embodiments. As is known in the art, the lead/lag reactor configuration in sequence enables improved isomerization through control of individual reactor temperatures and for partial catalyst replacement without a process shutdown. First isomerization reactor zone 106 operates by receiving the feed stream 105 into lead reactor 106A, wherein it is reacted at first reaction conditions to form isomerized paraffins. The product of lead reactor 106A, which leaves the reactor via stream 111, includes a combination of straight-chain paraffins, cyclo-paraffins, and isomerized paraffins. Stream 111 exchanges heat with the feed stream 105 in heat exchanger 107B, and is thereafter passed to lag reactor 106B. Lag reactor operates at second reactor conditions to form additional isomerized paraffins. The product of lag reactor 106B, which leaves the reactor via stream 113, includes a combination of straight-chain paraffins, cyclo-paraffins, and isomerized paraffins, with an increased percentage of isomerized paraffins as compared to the product of lead reactor 106B. Stream 113 exchanges heat with the feed stream 105 in heat exchanger 107A, and is thereafter optionally passed to a separator 108.

In the first isomerization reactor zone 106 the isomerization feed 105 is subjected to isomerization conditions including the presence of isomerization catalyst in the presence of a limited but positive amount of hydrogen as described in U.S. Pat. Nos. 4,804,803 and 5,326,296, both herein incorporated by reference. The isomerization of paraffins is generally considered a reversible first order reaction. Thus, the isomerization reaction product or effluent will contain a greater concentration of non-linear paraffins and a lesser concentration of linear paraffins and cyclo-paraffins than does the isomerization feed 105. In some embodiments, the isomerization conditions are sufficient to isomerize at least about 20, for example, between about 30 and about 60, mass-percent of the normal paraffins and cyclo-paraffins in the isomerization feed, between the lead and lag reactors 106A, 106B.

For example, the isomerization conditions in the first isomerization reactor zone 106 achieve at least about 70, such as at least about 75, or, from about 75 to about 97, percent of equilibrium for C₅ paraffins and C₆ paraffins present in the isomerization feed 105. In many instances, the isomerization reaction effluent has a mass ratio of non-linear paraffins to linear paraffins and cyclo paraffins of at about least 2:1, for example from about 2.5 to about 4:1.

The isomerization catalyst is not critical to the broad aspects of the processes of this disclosure, and any suitable isomerization catalyst may find application. Suitable isomerization catalysts include acidic catalysts using chloride for maintaining the sought acidity and sulfated catalysts. The isomerization catalyst may be amorphous, e.g. based upon amorphous alumina, or zeolitic. A zeolitic catalyst would still normally contain an amorphous binder. The catalyst may include a sulfated zirconia and platinum as described in U.S. Pat. No. 5,036,035 and European application 0 666 109 A1 or a platinum group metal on chlorided alumina as described in U.S. Pat. Nos. 5,705,730 and 6,214,764. Another suitable catalyst is described in U.S. Pat. No. 5,922,639. U.S. Pat. No. 6,818,589 discloses a catalyst including a tungstated support of an oxide or hydroxide of a Group IVB (IUPAC 4) metal, for example zirconium oxide or hydroxide, at least a first component which is a lanthanide element and/or yttrium component, and at least a second component being a platinum-group metal component. These documents are incorporated herein for their teaching as to catalyst compositions, isomerization operating conditions, and techniques.

Contacting within the first isomerization reactor zone 106 may be effected using the catalyst in a fixed-bed system, a moving-bed system, a fluidized-bed system, or in a batch-type operation. A fixed-bed system may be employed in an exemplary embodiment. The reactants may be contacted with the bed of catalyst particles in upward, downward, or radial-flow fashion. The reactants may be in the liquid phase, a mixed liquid-vapor phase, or a vapor phase when contacted with the catalyst particles. The first isomerization reactor zone 106 may include suitable means to ensure that the desired isomerization temperature is maintained at the entrance to each reactor 106A, 106B. As noted above, the exemplary lead/lag reactor configuration in sequence enables improved isomerization through control of individual reactor temperatures and for partial catalyst replacement without a process shutdown.

Isomerization conditions in the lead and lag reactors 106A, 106B include reactor temperatures that may be from about 40° C. to about 250° C. Lower reaction temperatures (within the stated range) may be employed in order to favor equilibrium mixtures having the highest concentration of high-octane highly branched isoalkanes and to minimize cracking of the feed to lighter hydrocarbons. Temperatures from about 100° C. to about 200° C. may be employed in some embodiments. Reactor operating pressures may be from about 100 kPa to about 10 MPa absolute, for example from about 0.5 to about 4 MPa absolute. Liquid hourly space velocities may be from about 0.2 to about 25 volumes of isomerizable hydrocarbon feed per hour per volume of catalyst, for example from about 0.5 to about 15 hr⁻¹.

Hydrogen is admixed with or remains with the isomerization feed to the isomerization reactors to provide a mole ratio of hydrogen to hydrocarbon feed of from about 0.01 to about 20, for example from about 0.05 to about 5. The hydrogen feed 104 may be supplied totally from outside the process (source 103) or, as shown in FIG. 1, supplemented by hydrogen recycled to the feed after separation from isomerization reactor effluent (described in greater detail below). Light hydrocarbons and small amounts of inerts such as nitrogen and argon may be present in the hydrogen. Water may be removed from hydrogen supplied from outside the process, for example by an adsorption system 101 as is known in the art.

Where a chlorided catalyst is used for isomerization, the isomerization reaction effluent may additionally contacted with a sorbent to remove any chloride components. Suitable systems therefor are disclosed in U.S. Pat. No. 5,705,730.

The effluent from the first isomerization zone 106 is optionally directed via line 113 to a separator 108, which may be configured as a physical packed-bed type separator configurated to separate the excess hydrogen, for example, in the product from the reactor zone 106. In one embodiment, the isomerization is conducted in the liquid phase and the isomerization reaction effluent is passed to separator 108 in which the gaseous overhead 115 containing hydrogen is obtained. At least a portion of this hydrogen may optionally be recycled to the isomerization reactor for providing at least a portion of the sought hydrogen for the isomerization. Accordingly, the hydrogen leaves the separator 108 via the overhead stream 115, where it joins with outside hydrogen feed 103, as initially noted above. In general, the isomerization reaction effluent may be subjected to one or more separation operations to provide a product fraction of an enhanced octane rating via stream 117 and, optionally, to remove other components such as hydrogen (via line 115), lower alkanes and, with respect to chlorided catalysts, halogen compounds. In embodiments where the separator 108 is not present, hydrogen may be removed (and not be recycled) using a stabilizer column as will be described in greater detail below.

The effluent from the separator is directed via line 117 to stabilizer column 110. In stabilizer column 110, light hydrocarbons (such as C₄ and lighter hydrocarbons, and possibly H₂ gas) are removed from an overhead portion of column 110 via line 119. The light hydrocarbons removed via line 119 may be used for any suitable purpose including for fuel value.

A product from a bottom portion of stabilizer column 110 exits the column through line 121, where it is cooled in heat exchanger 123 against a suitable cooling fluid or against a deisohexanizer bottom product stream (described below), and thereafter passed to a deisohexanizer (DIH) 112. The liquid bottom portion product in line 121 is passed to DIH 112 to provide a lower boiling fraction containing dimethylbutanes as a product from an overhead portion (stream 125) of the DIH 112 and a higher boiling fraction containing C₇ and heavier hydrocarbons as a product from a bottom portion (stream 129) of the DIH 112, for example. As shown in FIG. 1, the deisohexanizer 112 is also adapted to provide a side stream 127, which may include normal hexane, methylpentanes, and methylcyclopentane. The deisohexanizer 112 may be a packed or trayed column and typically operates with a top pressure of from about 50 to about 500 kPa (gauge) and a bottom temperature of from about 75° C. to about 170° C. The lower boiling fraction is provided from an overhead portion of DIH 112 via line 125. The higher boiling is provided form a bottom portion of DIH 112 via line 129. Lines 125 and 129 are optionally combined as product 114, or used individually as separate products. The side stream 127 from deisohexanizer 112 is thereafter passed via line 127 to a second isomerization reactor zone 206 using the action of pump 131, for example.

The composition of the lower boiling fraction from the deisohexanizer 112 will depend upon the operation and design of the assembly and any separation processes to which the isomerization effluent has been subjected. In some embodiments, the lower boiling fraction may contain about 20 to about 70 mass-percent dimethylbutanes, about 10 to about 40 mass-percent normal pentane, and about 20 to about 60 mass-percent isopentane and butane. Depending upon the operation of the deisohexanizer 112, the lower boiling fraction may also contain significant, for example, at least about 10 mass-percent methylpentanes.

The side-cut fraction contains normal hexane, methylpentanes, and methylcyclopentane. In some embodiments, the side-cut fraction may contain about 2 to about 10 mass-percent dimethylbutanes, about 5 to about 50 mass-percent normal hexane, about 20 to about 60 mass-percent methylpentanes, and about 5 to about 25 mass-percent methylcyclopentane. The deisohexanizer 112 may be designed to provide a side stream that contains methyl pentanes, methylcyclopentane, normal hexane, dimethylbutanes and cyclohexane. The bottom stream 129 contains cyclohexane and C₇ and heavier hydrocarbons.

Regarding stream 127, at least a portion, for example at least 50, such as at least 80, mass-percent to substantially all of the side-cut fraction from the deisohexanizer 112 is sent to the second isomerization reactor zone 206. Stream 127and hydrogen stream 104B thereafter combined into a feed stream 128. Feed stream 128 passes through two heat exchangers 207A, 207B (described in greater detail below) and a heater 209 to increase the temperature of the stream 128 to a suitable isomeration reactor temperature, as will be described in greater detail below. Upon passing through heat exchangers 207A, 207B, and heater 209, the stream 128 enters a lead isomerization reactor 206A of the second isomerization reactor zone 206. The second isomerization reactor zone 206 operates by receiving the stream 128 into lead reactor 206A, wherein it is reacted at first reaction conditions to form isomerized paraffins. The product of lead reactor 206A, which leaves the reactor via stream 211, includes a combination of straight-chain paraffins and isomerized paraffins. Stream 211 exchanges heat with the stream 128 in heat exchanger 207B, and is thereafter passed to lag reactor 206B. Lag reactor operates at second reactor conditions to form additional isomerized paraffins. The product of lag reactor 206B, which leaves the reactor via stream 213, includes a combination of straight-chain paraffins and isomerized paraffins, with an increased percentage of isomerized paraffins as compared to the product of lead reactor 206B. Stream 213 exchanges heat with the stream 128 in heat exchanger 207A, and is thereafter passed to a separator 108 by joining with stream 113 (isomerized product from first lead/lag reactor pair 106). As noted above with regard to first isomerization reactor zone 106, the second isomerization reactor zone 206 may alternatively include one, three, or more isomerization reactors.

In the isomerization reactor zone 206 the isomerization stream 128 is subjected to isomerization conditions including the presence of isomerization catalyst in the presence of a limited but positive amount of hydrogen as described in U.S. Pat. Nos. 4,804,803 and 5,326,296, both herein incorporated by reference. Thus, the isomerization reaction product or effluent will contain a greater concentration of non-linear paraffins and a lesser concentration of linear paraffins and cyclic paraffins than does the side-cut stream 127 from DIH 112. In some embodiments, the isomerization conditions are sufficient to isomerize at least about 20, for example, between about 30 and about 60, mass-percent of the normal paraffins and cyclo-paraffins in the isomerization stream 128, between the lead and lag reactors 206A, 206B. For example, the isomerization conditions in the lead/lag reactor pair 206 achieve at least about 70, such as at least about 75, or, from about 75 to about 97, percent of equilibrium for C₅ and C₆ paraffins present in the stream 128. In many instances, the isomerization reaction effluent has a mass ratio of non-linear paraffins to linear paraffins of at about least 2:1, for example from about 2.5 to about 4:1.

The isomerization catalyst is not critical to the broad aspects of the processes of this disclosure, and any suitable isomerization catalyst may find application. Suitable isomerization catalysts include acidic catalysts using chloride for maintaining the sought acidity and sulfated catalysts. The isomerization catalyst may be amorphous, e.g. based upon amorphous alumina, or zeolitic. A zeolitic catalyst would still normally contain an amorphous binder. The catalyst may include a sulfated zirconia and platinum as described in U.S. Pat. No. 5,036,035 and European application 0 666 109 A1 or a platinum group metal on chlorided alumina as described in U.S. Pat. Nos. 5,705,730 and 6,214,764. Another suitable catalyst is described in U.S. Pat. No. 5,922,639. U.S. Pat. No. 6,818,589 discloses a catalyst including a tungstated support of an oxide or hydroxide of a Group IVB (IUPAC 4) metal, for example zirconium oxide or hydroxide, at least a first component which is a lanthanide element and/or yttrium component, and at least a second component being a platinum-group metal component. These documents are incorporated herein for their teaching as to catalyst compositions, isomerization operating conditions, and techniques.

Contacting within the second isomerization reactor zone 206 may be effected using the catalyst in a fixed-bed system, a moving-bed system, a fluidized-bed system, or in a batch-type operation. A fixed-bed system may be employed in an exemplary embodiment. The reactants may be contacted with the bed of catalyst particles in upward, downward, or radial-flow fashion. The reactants may be in the liquid phase, a mixed liquid-vapor phase, or a vapor phase when contacted with the catalyst particles. The second isomerization reactor zone 206 may include suitable means to ensure that the desired isomerization temperature is maintained at the entrance to each reactor 206A, 206B.

Isomerization conditions in the lead and lag reactors 206A, 206B include reactor temperatures that may be from about 40° C. to about 250° C. Temperatures from about 100° C. to about 200° C. may be employed in some embodiments. The temperature requirement in second isomerization reactor zone 206 is lower than the first isomerization reactor zone 106, for example by about 10° C. to about 40° C. lower. This lower temperature further improves the equilibrium concentration towards highly branched isoalkanes and minimizes cracking of the hydrocarbons in the stream 128 to lighter hydrocarbons. Reactor operating pressures may be from about 100 kPa to about 10 MPa absolute, for example from about 0.5 to about 4 MPa absolute. Liquid hourly space velocities may be from about 0.2 to about 25 volumes of isomerizable hydrocarbon feed per hour per volume of catalyst, for example from about 0.5 to about 15 hr⁻¹.

Hydrogen is admixed with the stream 127 to the isomerization reactors 206 to provide a mole ratio of hydrogen to hydrocarbon of from about 0.01 to about 20, for example from about 0.05 to about 5. The hydrogen feed 104B, split from line 104, may be supplied totally from outside the process (source 103) or, as shown in FIG. 1, supplemented by hydrogen recycled to the feed after separation from isomerization reactor effluent (described in greater detail above). As noted above, the effluent from the second isomerization reactor zone 206 is directed via line 213 to join with line 113 prior to entry into the separator 108 (optionally), or into the stabilizer 110. The effluent from reactor pair 206 thus passes through the separator 108 (optionally), the stabilizer 110, and the DIH column 112 to form product 114 as described above.

### ILLUSTRATIVE EXAMPLE

The present disclosure is now illustrated by the following non-limiting example. It should be noted that various changes and modifications can be applied to the following example and processes without departing from the scope of this invention, which is defined in the appended claims. Therefore, it should be noted that the following example should be interpreted as illustrative only and not limiting in any sense.

Mathematical simulations were conducted to calculate the overall improvement of the embodiments described above, in comparison to a prior art example the employs hydrocarbon recycling to a single isomerization reactor zone. The “Base Case” in the following table includes only a first paired lead/lag reactor flow scheme where the dehexanizer column sidecut is processed in the first paired lead/lag reactors along with the freash feed. Table 1 below displays the improvement (increase) in isomerate yield and (decrease) catalyst quantity for the flow scheme of the described embodiments. It is also noted that the described embodiments require less H₂ feed.

TABLE 1

| | | Described |
|---|---|---|
| Parameters | Base Case | Embodiments |
| Product RON | Base | Base |
| Product MON | Base | Base |
| Isomerate Yield, wt % of | Base | Base + 0.6 to 1.0 |
| fresh feed | | |
| Total catalyst | Base | Base − 18% to 20% |
| Chemical H₂ consumption | Base | Base − 4% to 5% |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the application in any way. Rather, the foregoing detailed description will provide those skilled in the art with a convenient road map for implementing one or more embodiments, it being understood that various changes may be made in the function and arrangement of elements described in an exemplary embodiment without departing from the scope, as set forth in the appended claims.

What is claimed is:

1. A method for isomerizing paraffins comprising the steps of:
providing a hydrocarbon stream comprising linear paraffin compounds and cyclo-paraffin compounds;
passing the hydrocarbon stream to a first isomerization reactor zone to isomerize a first portion of the linear paraffin compounds and the cyclo-paraffin compounds to branched paraffin compounds, wherein a second portion of the linear paraffin compounds and the cyclo-paraffin compounds remain as linear paraffins compounds and cyclo-paraffins compounds;
deisohexanizing the branched and linear paraffin compounds to form an overhead product stream, a bottom product stream, and a side-cut stream compising the linear paraffin compounds; and
passing the side-cut stream to a second isomerization reactor zone to isomerize the linear paraffin compounds and cyclo-paraffin compounds to branched paraffin compounds.

2. The method of claim 1, further comprising passing hydrogen gas to both the first and second first and second reactor zones.

3. The method of claim 1, further comprising separating hydrogen gas from an effluent of the first and second reactor zones.

4. The method of claim 3, further comprising stabilizing the effluent of the first and second reactor zones.

5. The method of claim 1, wherein deisohexanizing comprises deisohexanizing the stabilized effluent of the first and second reactor zones.

6. The method of claim 1, wherein providing the hydrocarbon stream comprises providing a hydrocarbon stream comprising C₅ and C₆ linear and cyclo-paraffins.

7. The method of claim 6, wherein deisohexanizing to form the overhead product stream comprises forming a product stream comprising C₅ and C₆ branched paraffins.

8. The method of claim 7, wherein deisohexanizing to form the bottoms product stream comprises forming a product stream comprising C₇ and heavier hydrocarbons.

9. The method of claim 8, wherein deisohexanizing to form the side-cut stream comprises forming a side-cut stream comprising normal hexane.

10. The method of claim 8, further comprising combining the overhead product stream and the bottoms product stream to form a hydrocarbon product.

11. The method of claim 1, wherein one or both of the first and second isomerization reactor zones are provided in a lead/lag configuration, comprising two or three reactors in series.

12. A system for isomerizing paraffins comprising:
a first reactor zone that receives a hydrocarbon stream comprising linear paraffin compounds and generates and effluent comprising linear paraffin compounds, cyclo-paraffins compounds, and branched paraffin compounds;
a deisohexanizer that receives the effluent from the first and a second reactor zone and generates an overhead product stream, a bottoms product stream, and a side-cut stream comprising the linear paraffin compounds; and
a second reactor zone that receives the side-cut stream and generates branched paraffin compounds.

13. The system of claim 12, further comprising a separator that separates hydrogen gas from the reactor effluent.

14. The system of claim 13, further comprising a stabilizer that removes C₄ and lighter hydrocarbons from the effluent.

15. The system of claim 12, wherein the first reactor zone comprises a lead reactor in series with a lag reactor.

16. The system of claim 15, wherein the first reactor zone comprises three reactors in series.

17. The system of claim 15, wherein the second reactor zone comprises a lead reactor in series wath a lag reactor.

18. The system of claim 17, wherein the second reactor zone comprises three reactors in series.

19. The system of claim 12, further comprising a hydrogen gas feed source that supplies hydrogen gas to both the first and second lead/lag reactor pairs.

20. A method for isomerizing paraffins comprising the steps of:
providing a hydrocarbon stream comprising linear paraffin compounds and cyclo-paraffin compounds;
passing the hydrocarbon stream to a first isomerization reactor zone to isomerize a first portion of the linear paraffin compounds and the cyclo-paraffin compounds to branched paraffin compounds, wherein a second portion of the linear paraffin compounds and the cyclo-paraffin compounds remain as linear paraffins compounds and cyclo-paraffins compounds;
deisohexanizing the branched and linear paraffin compounds to form an overhead product stream, a bottom product stream, and a side-cut stream comprising the linear paraffin compounds;
passing the side-cut stream to a second isomerization reactor zone to isomerize the linear paraffin compounds and cyclo-paraffin compounds to branched paraffin compounds;
passing hydrogen gas to both the first and second reactor zones; and
separating hydrogen gas from an effluent of the first and second reactor zones.
